(19) United States
(12) Patent Application Publication
(10) Pub. No.: US 2015/0196239 A1
(43) Pub. Date: Jul. 16, 2015

(54) METHOD AND APPARATUS FOR DRIVING AN EMITTER IN A MEDICAL SENSOR

(71) Applicant: Covidien LP, Mansfield, MA (US)

(72) Inventors: Christopher J. Meehan, Golden, CO (US); Daniel Lisogurski, Boulder, CO (US); Jonathan Snyder, Northglen, CO (US); William Kit Dean, Castle Pines, CO (US); Friso Schlottau, Lyons, CO (US)

(21) Appl. No.: 14/572,283

(22) Filed: Dec. 16, 2014

Related U.S. Application Data
(60) Provisional application No. 61/926,105, filed on Jan. 10, 2014.

(51) Int. Cl. A61B 5/1455; A61B 5/024

(52) U.S. Cl. CPC A61B 5/14552; A61B 5/02416

(57) ABSTRACT

A oximetry device includes a controlled switch configured to transmit and resist a current flow. The oximetry device also includes an energy storage element configured to store energy in response to the current flow being transmitted and discharge the stored energy to a diode as a diode current in response to the controlled switch resisting the current flow. The oximetry device further comprises a sense resistor configured to receive diode current and generate a sensed voltage based on the diode current. The oximetry device additionally comprises and a comparator configured to activate the controlled switch to transmit the current flow and deactivate the controlled switch to resist the current flow based upon the sensed voltage.

### CROSS REFERENCE TO RELATED APPLICATIONS

This application claims the benefit of U.S. Provisional Patent Application No. 61/926,105, filed Jan. 10, 2014.

### BACKGROUND

The present disclosure relates generally to medical devices and, more particularly, to pulse oximetry systems used for sensing physiological parameters of a patient.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. One or more of the above physiological characteristics may then be calculated based upon the amount of light absorbed or scattered. More specifically, the light passed through the tissue is typically selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

The light sources utilized in pulse oximeters typically must be driven based on various parameters including their ability to transmit light at specific wavelengths so that the absorption and/or scattering of the transmitted light in a patient's tissue may be properly determined. However, there are other concerns that accompany the riving of the light sources, such as accuracy and power consumption. For example, when a pulse oximeters is being driven by a local power source (e.g., a battery), reduced power consumption results in longer battery life. However, coupled with this desire for power consumption and efficiency during the operation of a pulse oximeters is this desire for accurate results to be generated. Thus, is desirable to increase the accuracy of while also increasing the efficiency of pulse oximeters.

### BRIEF DESCRIPTION OF THE DRAWINGS

Advantages of the disclosure may become apparent upon reading the following detailed description and upon reference to the drawings in which:

FIG. 1 illustrates a perspective view of a pulse oximeter in accordance with an embodiment;

FIG. 2 illustrates a simplified block diagram of a pulse oximeter of FIG. 1, in accordance with an embodiment;

FIG. 3 illustrates a simplified block diagram of a regulator of current to an emitter of the pulse oximeter of FIG. 1, in accordance with an embodiment;

FIG. 4 illustrates a simplified block diagram of a portion of the regulator of FIG. 3, in accordance with an embodiment;

FIG. 5 illustrates a second simplified block diagram of a portion of the regulator of FIG. 3, in accordance with an embodiment;

FIG. 6 illustrates a simplified block diagram of filtering circuitry for use with a sensor of the pulse oximeter of FIG. 1, in accordance with an embodiment;

FIG. 7 illustrates a simplified block diagram of a sensor of the pulse oximeter of FIG. 1, in accordance with an embodiment;

FIG. 8 illustrates waveforms relating to the operation of a sensor of the pulse oximeter of FIG. 1, in accordance with an embodiment;

FIG. 9 illustrates second waveforms relating to the operation of the sensor of the pulse oximeter of FIG. 1, in accordance with an embodiment; and

FIG. 10 illustrates waveforms relating to the operation of the sensor of FIG. 7, in accordance with an embodiment;

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present application sets forth the use of a switching regulator (e.g., a Switch Mode Power Supply or SMPS), such as a buck, boost, buck/boost, or charge pump regulator for use in conjunction with a medical device, such as a pulse oximeter. The regulator may operate with the feedback point coming from the current through a light source such as an LED. This may include creating a current mode switching regulator that can be driven hysteretically or with a continuous frequency. Additionally, the efficiency of the switching regulator is much higher than traditional linear mode LED drive because power is not wasted in via a voltage drop across the transistor. Instead by using a switching regulator, the power is stored in the inductor as flux (rather than converting it to heat) and then can be converted back into the desired current as determined by the duty cycle of the switching regulator.

In some embodiments, the current may be filtered before going to the LED such that the LED has less variation in intensity. This filtering also reduces high frequency emissions from the sensor cable. Additionally, a switching regulator technique may be utilized to send pulses of power into an inductor such that as the inductor discharges, it is forced to pass current through the LED and the rest of the circuit (inclusive of a feedback resistor), which may be used as an indicator of how much current is passed through the LED. A feedback circuit of the regulator determines whether to add more or less current into the LED to allow for precise control over current into the LED, while wasting minimal energy.

Additionally, for the pulse oximeter to be useable for a large variety of patients, a wide range of LED drive currents may be implemented. Accordingly, techniques and circuits are set forth that allow for multiple and/or variable resistances to be present as the feedback resistor. Furthermore, non-circuit solutions for reduction of energy and/or an increase in the reliability of measured signals for pulse oximeters may be present in the certain embodiments. For example, a photo detector output as well as an LED drive current may be sampled and utilized to derive or look up an offset coefficient that may be applied to the received signals from the photo detector to correct for errors in the received signals prior to or in conjunction with the calculation of physiological parameters of a patient.

Moreover, the present application details filtering techniques whereby each LED of a sensor of a pulse oximeter corresponds to a particularly tuned filter. By implementing particularized filters, advantages in signal quality may be achieved. Also set forth in the present application is a technique that allows for differential driving of LEDs of a sensor of a pulse oximeter so that greater signal quality in conjunction with reduced circuitry and components (e.g., cable shielding) may be realized.

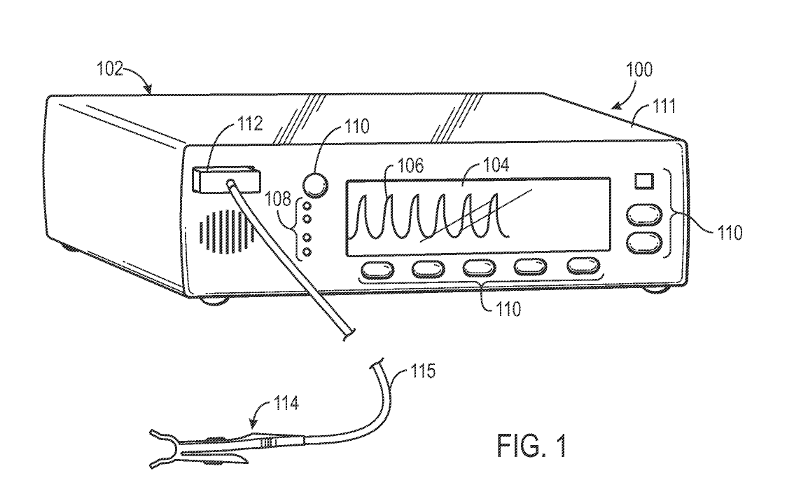

Turning to FIG. 1, a perspective view of a medical device is illustrated in accordance with an embodiment. The medical device may be a pulse oximeter 100. It should be noted that the medical device may also be another type of device that measures physiological parameters of a patient, such as a regional oximeter or other device. The pulse oximeter 100 may include a monitor 102, such as those available from Nellcor Puritan Bennett LLC. The monitor 102 may be configured to display calculated parameters on a display 104. As illustrated in FIG. 1, the display 104 may be integrated into the monitor 102. However, the monitor 102 may be configured to provide data via a port to a display (not shown) that is not integrated with the monitor 102. The display 104 may be configured to display computed physiological data including, for example, an oxygen saturation percentage, a pulse rate, and/or a plethysmographic waveform 106. As is known in the art, the oxygen saturation percentage may be a functional arterial hemoglobin oxygen saturation measurement in units of percentage Sp_{O2}, while the pulse rate may indicate a patient's pulse rate in beats per minute. The monitor 102 may also display information related to alarms, monitor settings, and/or signal quality via indicator lights 108.

To facilitate user input, the monitor 102 may include a plurality of control inputs 110. The control inputs 110 may include fixed function keys, programmable function keys, and soft keys. Specifically, the control inputs 110 may correspond to soft key icons in the display 104. Pressing control inputs 110 associated with, or adjacent to, an icon in the display may select a corresponding option. The monitor 102 may also include a casing 111. The casing 111 may aid in the protection of the internal elements of the monitor 102 from damage.

The monitor 102 may further include a sensor port 112. The sensor port 112 may allow for connection to an external sensor 114, via a cable 115 which connects to the sensor port 112. The sensor 114 may be of a disposable or a non-disposable type. Furthermore, the sensor 114 may obtain readings from a patient, which can be used by the monitor to calculate certain physiological characteristics such as the blood-oxygen saturation of hemoglobin in arterial blood, the volume of individual blood pulsations supplying the tissue, and/or the rate of blood pulsations corresponding to each heartbeat of a patient. In some embodiments, the sensor 114 may be a wireless sensor capable of wirelessly communicating with the monitor 102 without the use of cable 115. However, regardless of whether the sensor 114 is wired or wireless, similar measurements may be monitored by and transmitted from the sensor 114 to the monitor 102 for determinations of physiological parameters of a patient.

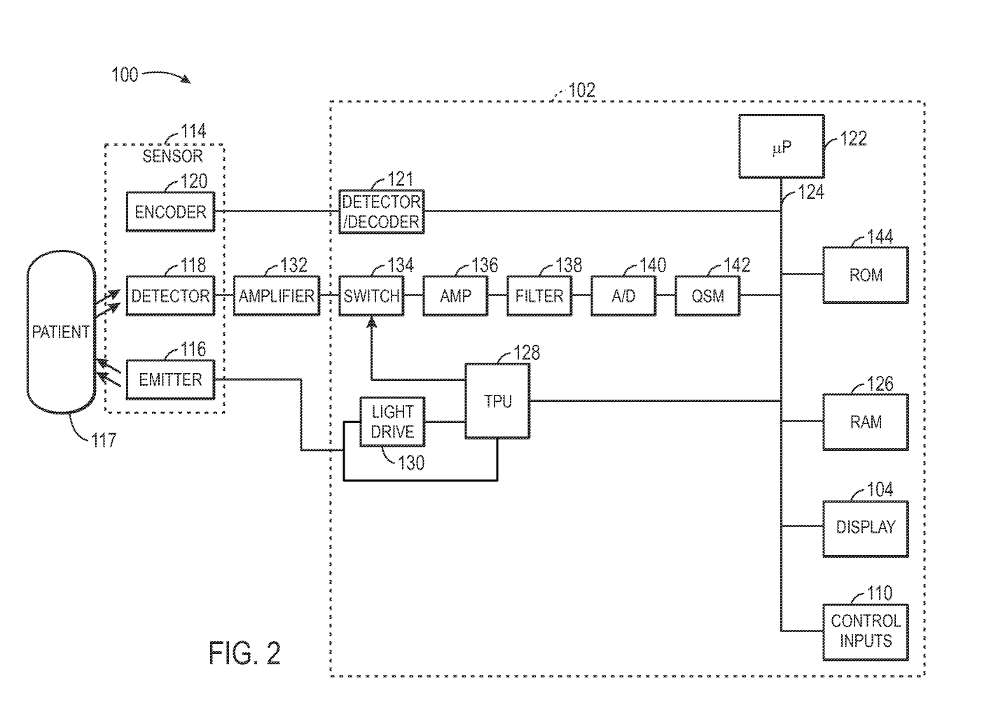

Turning to FIG. 2, a simplified block diagram of a pulse oximeter 100 is illustrated in accordance with an embodiment. Specifically, certain components of the sensor 114 and the monitor 102 are illustrated in FIG. 2. The sensor 114 may include an emitter 116, a detector 118, and an encoder 120. It should be noted that the emitter 116 may be capable of emitting at least two wavelengths of light, e.g., RED and infrared (IR) light, into the tissue of a patient 117 to allow for calculation of physiological characteristics of a patient 117, where the RED wavelength may be between about 600 nanometers (nm) and about 700 nm, and the IR wavelength may be between about 800 nm and about 1000 nm. A single broadband light source may be used as the emitter 116 or, alternatively, two separate light sources (e.g., LEDs) may be utilized whereby each light source transmitting light at a particular wavelength, including the RED and IR wavelengths. The light may be transmitted into a patient 117 for use in measuring, for example, water fractions, hematocrit, or other physiologic parameters of the patient 117. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In one embodiment, the detector 118 may be capable of detecting light at various intensities and wavelengths. In operation, light enters the detector 118 after passing through the tissue of the patient 117. For example, light from the emitter 16 may pass into a blood perfused tissue of the patient 117, may be scattered, and then detected by one or more detectors 118. The detector 118 may convert the light at a given intensity, which may be directly related to the absorbance and/or reflectance of light in the tissue of the patient 117, into an electrical signal. That is, when more light at a certain wavelength is absorbed or reflected, less light of that wavelength is typically received from the tissue by the detector 118. After converting the received light to an electrical signal, the detector 118 may send the signal to the monitor 102, where physiological characteristics may be calculated based at least in part on the absorption of light in the tissue of the patient 117.

Additionally the sensor 114 may include an encoder 120, which may contain information about the sensor 114, such as what type of sensor it is (e.g., whether the sensor is intended for placement on a forehead or digit) and the wavelengths of light emitted by the emitter 116. This information may allow the monitor 102 to select appropriate algorithms and/or calibration coefficients for calculating the patient's physiological characteristics. The encoder 120 may, for instance, be a memory on which one or more of the following information may be stored for communication to the monitor 102: the type of the sensor 114; the wavelengths of light emitted by the emitter 116; and the proper calibration coefficients and/or algorithms to be used for calculating the patient's 117 physiological characteristics.

Signals from the detector 118 and the encoder 116 may be transmitted to the monitor 102. The monitor 102 may include one or more processors 122 coupled to an internal bus 124. Also connected to the bus may be, for example, a RAM memory 126, the display 104, control inputs 110, and a decoder 121, which may receive signals from encoder 120 and may transmit an indication of those signals to the processor(s) 122 to allow for determination of proper calibration coefficients and/or algorithms to be used for calculating the patient's 117 physiological characteristics.

Additionally, the monitor 102 includes a time processing unit (TPU) 128 coupled to the one or more processors 122. The TPU 128 may provide timing control signals to light drive circuitry 130, which controls when the emitter 116 is activated and, if multiple light sources are used, the multiplexed timing for the different light sources. TPU 128 may also control the gating-in of signals from detector 118 through an amplifier 132 and a switching circuit 134. These signals are sampled at the proper time, depending at least in part upon which of multiple light sources is activated, if multiple light sources are used. The received signal from the detector 118 may be passed through an amplifier 136, a low pass filter 138, and an analog-to-digital converter 140 for amplifying, filtering, and digitizing the electrical signals the from the sensor 114. The digital data may then be stored in a queued serial module (QSM) 142, for later downloading to RAM 126 as QSM 142 fills up. In an embodiment, there may be multiple parallel paths of separate amplifier, filter, and A/D converters for multiple light wavelengths or spectra received.

In an embodiment, based at least in part upon the received signals corresponding to the light received by detector 118, processor 122 may calculate the oxygen saturation using various algorithms. These algorithms may require coefficients, which may be empirically determined, and may correspond to the wavelengths of light used. The algorithms may be stored in a ROM 144 and accessed and operated according to processor 122 instructions. For example, the encoder 120 may communicate with decoder 121 to provide information for used by the processor 122 to determine the appropriate coefficients.

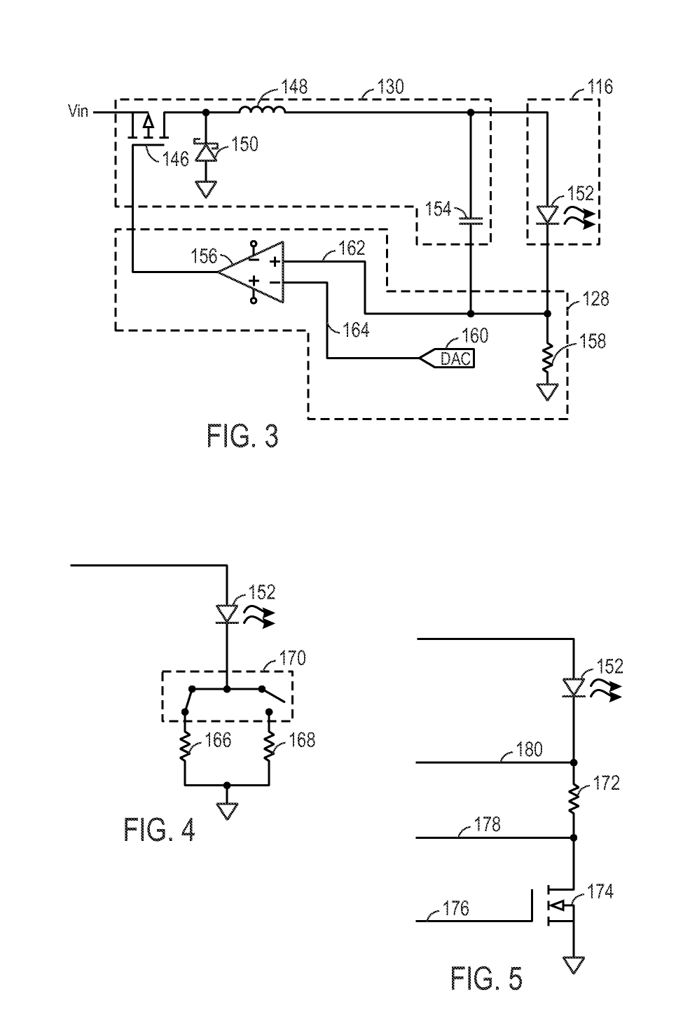

FIG. 3 illustrates an embodiment that may include various elements of the monitor 102 and the sensor 114 and illustrates the interaction of these elements. As illustrated in FIG. 3, the TPU 128 is coupled to the light drive circuitry 130 as well as the emitter 116. The interaction of the TPU 128, the light drive circuitry 130, and the emitter 116 is as follows. As controlled switch 146 (e.g., a transistor, a switching element, or other switching component able to break and couple the electrical circuit) is switched on, the inductor 148 draws current and begins to charge from voltage source Vin. Additionally present may be a diode 150 that does not conduct under normal conditions, but rather when driving current of the inductor is interrupted, the diode 150 enters into forward conduction mode, allowing the stored energy of the inductor 148 to be dissipated. In some embodiments, the diode 150 may be a Schottky diode to allow for rapid switching speeds.

Once the controlled switch 146 is turned off, the energy built up in the inductor 148 begins to discharge. As the inductor 148 discharges, it is forced to pass current through the diode 152 of the emitter 116, as well as through capacitor 152. In some embodiments, the diode 152 may be a light emitting diode capable of broadband light emission or a light emitting diode capable of emitting light at a particular wavelength (e.g., RED light or IR light). Additionally, while a single LED 150 is illustrated as being present in the emitter 116, this is for illustrative purposes only, since it is envisioned that one or more additional LEDs may also be present in the emitter 116 and controlled in a similar manner to that discussed herein with respect to diode 152.

The capacitor 154 assists in maintaining the current output by the inductor 148 at a substantially constant level. In this manner, the capacitor 154 operates as a filter. Additionally, while capacitor 154 is illustrated as being located in the light drive circuitry 130, in some embodiments, the capacitor 154 may be located in the sensor 114. By placing the capacitor 154 in parallel with the diode 152, increased noise rejection may be attained for the system (i.e., allowing a less noisy signal to be transmitted to the input of the switching comparator 156). For example, this configuration of the capacitor 154 in parallel with the diode 152 may allow for filtering of a potential current ripple prior to the current being transmitted to the diode 152, that reducing variation in intensities of the diode 152. Thus, the capacitor 154 operates to bypass high frequency noise away from the diode 152, and allows the currents transmitted to the diode 152 to be a cleaner (e.g., less noisy) signal than would otherwise be present if the capacitor 154 was not utilized as described above. This filtering performed by the capacitor 154 may also operate to reduce high frequency emissions from, for example, the sensor cable 115.

Additionally illustrated in FIG. 3 is a sense resistor 158. This sense resistor 158 may receive current that has passed through the diode 152. In some embodiments, the resistor 158 may be used as an indication as to how much current has passed through the diode 152. For example, the current through the resistor 158 may be converted to a voltage to be measured by the switching comparator 156 (e.g., a feedback circuit) that allows for the addition or reduction of current being transmitted to the diode 152. This use of a feedback loop allows precise control over current into the diode 152 while reducing wasted energy and, thus, may be beneficially utilized in conjunction with portable and/or battery operated pulse oximeters 100. Additionally, it should be noted that the sense resistor 158 may be present in the sensor 114 instead of in the TPU 1 sense resistor 158.

Furthermore, while it is illustrated that the switching comparator 156 is located in the TPU 128, in at least one embodiment, the comparator 156 may instead be part of processor 122. In addition, the illustrated digital to analog converter (DAC) 160 may also be present as part of the processor 122. That is, in some embodiments, the processor 122 may allow for the application of various filter and hysteresis settings, which may be controlled by software stored in a tangible non-transitory computer readable medium such as RAM 122 and/or ROM 144 for execution by the processor 122. In some embodiments, the programmable settings executable by the processor 122 may allow, for example, for the alteration of a frequency content of the signals transmitted to the diode 152. For example, through changing the frequency of the signals (e.g., current) transmitted to the diode 152, overall drive emissions of the emitter 116 may be spread over a wider frequency range, thus reducing interference and/or emissions at any one particular frequency. In some embodiments, the current output may be set by the DAC 160 communicatively coupled to the switching comparator 158.

For example, when the measured current received along path 162 is lower than a set point value transmitted along path 164, the comparator 156 activates the controlled switch 146 to allowing current to flow through the inductor 148 and, thus, the diode 152. In some embodiments, the comparator 156 input may receive an offset signal along path 164 from the DAC 160 that is non-zero while still allowing for disabling of the regulator circuitry of the diode 152 (e.g., switch 146, inductor 148, capacitor 154, and/or resistor 158) during diode 152 off periods. This is advantageous because most DACs 160 have an offset voltage and, thus, the signal transmitted along path 164 will not typically be able to be set to exactly zero. Other techniques for disabling the regulator circuitry of the diode 152 during diode 152 dark times may include, for example, by using a switch along path 164, disabling the comparator 156 and holding the output thereof in the off state, or by using a pulse width modulator or other circuit to generate a square wave in place of one or both of the comparator 156 and the DAC 160.

When the DAC 160 is utilized, the DAC 160 may be calibrated such that known settings of the DAC 160 will correspond to particular currents provided to the diode 152. This calibration may be performed, for example, utilizing a program stored in a tangible non-transitory medium (e.g., RAM 126 or ROM 144) and executed by the processor 122. For example, when a sensor 114 is connected to the monitor 102, the processor 122 may execute a program that initialize the DAC 160 to a first setting that causes a first signal to be transmitted along path 164. The current produced in connection with the diode 152 with this setting may be determined by the processor 122 and stored, for example, in RAM 126. The processor 122 may then continue to execute the DAC 160 initialization program by setting the DAC 160 to a second setting that causes a second signal to be transmitted along path 164. The current produced in connection with the diode 152 with this second setting may be determined by the processor 122 and stored, for example, in RAM 126.

Both a DAC 160 offset value and a slope corresponding to change in the DAC 160 settings vs. measured current to the diode 152 may be calibrated from these two readings. In some embodiments, the current produced in connection with the diode 152 may be measured by the processor 122 as based on a voltage drop across a known resistance when driving the diode 152. This calibration may be performed at the factory and stored, for example, in the encoder 120, RAM 126, or the ROM 144. This calibration also may be performed upon initialization (turning on) of the pulse oximeter 100. Additionally, in some embodiments, the slope and offset of the DAC 160 may be calculated separately using a calibration for each diode 152 present in the emitter 116 (e.g., an IR light emitting diode or a RED light emitting diode), since the offset voltages for a different diodes 152 may vary.

In some embodiments, diode 152 may be at least one light source driven over a range of currents. For example, the pulse oximeter 100 drives a RED light emitting diode (LED) and an IR LED. Typically, the driving of the diode 152 is such that the driving of the diode 152 has increased efficiency at certain current levels (e.g., low current levels) or and increased accuracy at other current levels (e.g., high current levels).

As noted above, the diode 152 drive current passes through a sense (feedback) resistor 158. Since V=IR, the voltage drop across the resistor 158 is determined by the chosen resistance value and by the diode 152 current. For example, if the resistance of resistor 158 is 1 Ohm and the current through the diode 152 is 50 mA then the voltage drop across the resistor is 50 mV. Furthermore, the comparator 156 may monitor this voltage value (e.g., voltage drop) to ensure the diode 152 current is correct and may also use this value as the feedback input to drive the desired current though the diode 152.

As power=VI=I²R, for the example above, the power dissipation in the resistor 158 is 0.050 A*0.050 A*1 Ohm=0.0025 W (2.5 mW). Increasing the resistance of resistor 158 to, for example, 10 Ohms would provide a higher voltage, thus increasing the accuracy of the determination of the current flowing to the diode 152 by providing more significant bits for an ADC sampling the voltage, and thus, changes thereof more easily detectable. However, this increase in the resistance of resistor 158 would also increase the power consumption by a factor of 10 from 2.5 mW to 25 mW. This accompanying increase in power consumption may be unacceptable for a low power pulse oximeter 100 (or a portable or battery operated oximeter 100). Thus, a tradeoff may occur of power consumption vs. accuracy in relation to the resistor 158.

Additionally, for the pulse oximeter 100 to be useable for a large variety of patients, a wide range of diode 152 drive currents may be implemented, further compounding the difficulties of this potential accuracy/power consumption tradeoff. Additionally, in many cases, the red LED will run at higher current that the IR LED so a single resistance for resistor 158 may not provide desired results. However, techniques presented in conjunction with FIGS. 4 and 5 allow for improved performance (e.g., efficiency and accuracy) across a range of supplied currents.

FIG. 4 illustrates one embodiment whereby the sense resistor 158 of FIG. 3 is replaced with a resistance value which can be changed. For example, one of N resistors may be selected by a switch or analog multiplexor. Thus, as illustrated in FIG. 4, sense resistors 166 and 168 may be utilized in place of sense resistor 158 of FIG. 3. Additionally, switch 170 may be utilized to activate and deactivate the connection path between each of sense resistors 166 and 168 and diode 152. In this manner, sense resistors 166 and 168 may be operated in parallel.

Moreover selecting suitable values for sense resistors 166 and 168 allows two settings to be selected for use with high or low currents. For example, if a low current is being utilized (e.g., approximately 3 mA), then a larger resistance value may be utilized. However, if a high current is being utilized (e.g., approximately 50 mA), then a smaller resistance value may be utilized. In this manner, the resistances of sense resistors 166 and 168 may differ from one another (i.e., one resistance may be greater than another) and switch 170 may select which sense resistor 166 or 168 is to be coupled to the diode 152 based on the current passing from the diode 152. The determination of when a large current or a small current is being utilized may be based on a number of factors including the location of the sensor 114 on a patient 117, the darkness of the skin of a patient 117, and/or other factors. For example, light skinned patients may require less current to be passed through diode 152 to generate usable signals for detection relative to darker skinned patients. By using multiple sense resistors 166 and 168, the overall power consumed may be matched to the patient 117 being monitored and, thus, may be more efficient and allow for greater reliability.

In other embodiments, one of sense resistors 166 and 168 may be eliminated and the internal resistance of the switch 170 may be utilized in place of one of sense resistors 166 or 168. Moreover, if the exact resistance of the switch 170 is not known (e.g., from a data sheet or other resource), the resistance of the switch 170 can be calibrated (for example, at power up or after a temperature change) by setting a diode 152 drive current that does not saturate either sense resistor 166 or 168 and comparing the known resistance values of sense resistors 166 or 168 with the switch open to when the switch 170 is closed. Moreover, the current passing through the diode 152 may, in one embodiment, be measured by means of a Hall Effect device, based on the magnetic field effect that the diode 152 current produces. In another embodiment, a digitally controlled potentiometer may be utilized in place of sense resistor 158 of FIG. 3.

Control over the switch 170 may come from a micro-controller of FPGA or other control logic present in TPU 128 or from processor 122. Moreover, in some embodiments, the micro-controller used to control the switch 170 may use a pulse width modulator to control switches in the emitter 116 that allows for the selection of the, for example, red and IR LED diodes in the emitter 116. If a PWM channel is also used for the sense resistor switch 170, it may also allow for different resistors to be used for different LED drive periods. For example, the IR LED in the emitter 116 may be driven at high current while the IR LED is driven at much lower current.

In another embodiment, a control line for switch 170 may be switched much faster than the diode 152 current changes. For example, if the switch 170 is pulsed (e.g., at a frequency of approximately 100 kHz), then the effective resistance will vary with the duty cycle of the switch 170 control line. This allows for the generation variable resistances, whereby the resistances are based on the pulsing characteristics of the switch 170. In this manner, through active control of the switch 170, the switch emulates the operation of a variable resistor but at low cost and complexity.

In another embodiment, the control line of the switch 170 may be switched more rapidly than the diode 152 current is typically changed for normal operation. For example, if the control line of the switch 170 is momentarily pulsed at a high frequency, then the current can be measured for very accurate calibration purposes, with the information obtained used to set variable parameters of the voltage regulator of FIG. 3 circuit to particular values that increase efficiency of the pulse oximeter 100 (e.g., optimum values). The control line can subsequently be returned to normal operation with an optimized value for the particular diode 152 in use. This may allow for increases in patient safety in monitoring the diode 152 current since an expected ratio of the switch 170 open and closed can be measured. If this ratio varies by more than the expected change in the resistance of, for example, the switch 170, then the system can detect a fault in current monitoring or drive and shut down.

Another technique for increasing the accuracy may include amplifying the output of sense resistors 166 and 168 (or resistor 158). For example, an operational amplifier may have its positive terminal coupled between switch 170 and each of sense resistors 166 and 168 and its negative terminal coupled between switch each of sense resistors 166 and 168 and ground. In this manner, the output of each of sense resistors 166 and 168 may be amplified to become an amplified output, which may then be monitored and fed back as a control signal, for example, along path 162 of FIG. 3. This amplification may increase the accuracy of monitoring and also reduces the regulator noise and/or errors.

FIG. 5 illustrates an additional technique to generate a variable resistance. As illustrated in FIG. 5, a resistor 172 (similar or identical to the sense resistor 158 of FIG. 3) may be utilized to calibrate a transistor 174 to operate as a variable resistor. In this embodiment, the resistor 172 has the same current passing through it as the transistor 174, which may be an NMOS transistor. Since a strong current signal may be desirable, for example, to allow for increased accuracy in patient 117 monitoring, an input control voltage on path 176 may be adjusted so that the current signal along path 178 is optimized to reach a threshold desired level for accuracy, thus reducing power consumption that would otherwise occur if a higher current were being transmitted along path 178. As this set point is reached, the control voltage on path 176 is fixed. This allows for a current measurement through resistor 172 may be measured by determining the voltage difference along paths 180 and 178. Additionally, once this current is determined, the resistance of the transistor 174 can be calculated as part of a calibration process whereby during normal operation of the pulse oximeter 100, accurate current measurements may be generated with while reducing or minimizing power wasted.

In another embodiment, the characteristic change in drain source impedance of a field effect device based on applied gate voltage may be used as the element of control to feed back to the switching regulator along path 162. In this embodiment, the applied gate control voltage can be switched based on whether, for example, a red LED or an IR LED is currently being used in the emitter 116. The switching of the gate control voltage can be made using capacitors to store the voltage that is used to bias the field effect device. The transient losses in changing the applied gate voltage can also be reduced by conserving the charge flowing between these capacitors in a reservoir or reservoirs. This conservation of flowing charge may further use transmission lines to ensure either voltages or currents (but not both) have finite values, so as to reduce switching losses.

In a patient 117 where the light has very small changes in attenuation over time (e.g., 0.03% modulation in very weak signals or in a patient 117 with low perfusion) the current noise (e.g., 1% error) may contribute significantly to the received signal emitted from diode 152. As such, reducing the current noise has several advantages including better signal to noise ratio and the ability to reduce diode 152 power, while still receiving acceptable signal levels.

However, there may be power, cost or size advantages to designing a noisy current source. Thus, instead of eliminating via design a noisy current source, actual currents generated over time may be measured and a correction factor that essentially removes or reduces the noise present in the processed signals may be included in the pulse oximeters 100. In one embodiment, a micro-controller, for example, processor 122, may be coupled to two independent ADC (analog to digital conversion) channels whereby both ADCs operate in the same mode of operation so that conversion time by the ADCs is identical. For example, the ADCs may both be differential inputs with 16-bit resolution. Both ADCs may use a common clock source as the conversion trigger start signal. In other embodiments, the triggered start of one ADC may be delayed relative to the other triggered start of the corresponding ADC to compensate for delays present in the system.

The ADCs may be discrete parts that may be set up in the same manner. Additionally, ADCs may alternatively be set up differently; however, this may require additional elements or design to achieve corrections similar to those generated via simultaneous sampling using similarly set up ADCs. Additionally, alternate methods and techniques for measuring or estimating currents are also contemplated, such as voltage to frequency converters or a DAC (digital to analog converter) used in conjunction with a comparator.

Particularly, the present technique may include sampling the photo detector 118 output, generally after signal conditioning such as amplification by, for example, amplifier 134 and/or amplifier 136 as well as after filtering by, for example, filter 138. The technique may also include sampling the diode 152 drive current and correcting the sampled amplitude of the photo detector 118 based on the measured current during each sample. Additionally, the technique may allow for a group delay correction to time align the diode 152 drive current in time with the output of the photo detector 118. For example, this delay correction may account for the group delay of filters in the photo detector 118 signal chain as well as different (or no) filtering in the monitor 102.

That is, the above discussed technique may be useful to correct errors in received photo detector 118 signals where an average current error occurs over a relatively short time period (e.g., 400 uS long LED pulse). The correction may be applied as follows:

$$f ( x ) = {\sum }_{n = 1}^{N} \frac{{P}_{n} \times {V}_{Target}}{{C}_{n}}$$

This represents f(x), whereby f(x) is the sum of the received signal over N samples (e.g., the samples where a particular LED is turned on), Pn is the photo detector 118 current at sample n, Cn is the measured diode 152 drive current at sample n, and Ctarget is the target drive current for diode 152 at sample N. This equation may be simplified as:

$$f ( x ) = {C}_{Target} \times {\sum }_{n = 1}^{N} \frac{{P}_{n}}{{C}_{n}}$$

Additionally, the current correction can be ignored during dark times, as the diode 152 is off. However, in the case of a system with an analog high pass filter, dark levels may be affected by the diode on times so the dark levels also may be corrected using the same values as on time values. In one embodiment, the above equation may be implemented fully by, for example, utilizing a program stored in a tangible non-transitory medium (e.g., RAM 126 or ROM 144) and executed by the processor 122. In another embodiment, the equation above may be approximated and the processor 122 may calculate calibration coefficients based on the approximation in place of the equation. This technique also may be implemented by, for example, utilizing a program stored in a tangible non-transitory medium (e.g., RAM 126 or ROM 144) and executed by the processor 122.

In one embodiment, the numerical approximation may exploit that Cn=Ctarget, such that Cn=Ctarget+CErrorn, where CErrorn equals the error in measured current at the diode 152 at time n and Cerrorn<<Ctarget. In one embodiment, a lookup table may be employed in the RAM 126 or ROM 144 to aid in speed of processing the calibration technique. For example, if Cn is derived from an 8-bit ADC, a 256 pt lookup table may be pre-loaded with the reciprocal inf, 1/1, 1/2, 1/3, . . . , 1/255 and the received values Pn can be multiplied by the entry table[Cn] to avoid additional processing in processor 122.

Similarly, one embodiment may utilize the form:

$$\frac{1}{1 - x} = 1 + x + {x}^{2} + {x}^{3} + {x}^{4} + {x}^{5} + … for = 1 < x < 1$$

to simplify the above equations for ease of implementation in the processor 122. That is, since fractions tend to require more processor cycles (are more power intensive to determine), the above noted approximation may be implemented by the processor 122 over the set of measured errors to simplify the calculations to:

$$\begin{matrix}f ( x ) = {C}_{Target} \times {\sum }_{n = 1}^{N} \frac{{P}_{n}}{{C}_{n}} \\ = {C}_{Target} \times {\sum }_{n = 1}^{N} \frac{{P}_{n}}{{C}_{Target} + {CError}_{n}} \\ = {P}_{n} \times {\sum }_{n = 1}^{N} \frac{1}{1 + \frac{{CError}_{n}}{{C}_{Target}}} \\ \approx {P}_{n} \times {\sum }_{n = 1}^{N} 1 - \frac{{CError}_{n}}{{C}_{Target}}\end{matrix}$$

Since Cerrorn<<Ctarget, Cerrorn÷Ctarget<0.01<<1, over the range of Target current errors of +/−1%, the resulting correction will have an error of less than 0.0001% relative to the fully implemented calculation. In other embodiments, the diode 152 may not be sampled, but instead an analog correction may be applied to the received signal. For example, the gain of an op-amp may vary based on the received current to apply a correction or a multiplying DAC may also be suitable to implement this analog correction.

Using the techniques described above, a correction factor may be determined by the processor 122 and applied to any measurements made of a patient 117. That is, photo diode 118 measurements may be sampled in conjunction with diode 152 drive current values. Based on these samples, the processor 122, utilizing the techniques discussed above, may determine errors present in the sampled signals and may apply a correction factor so that the psychological data of a patient 117 may more accurately be determined.

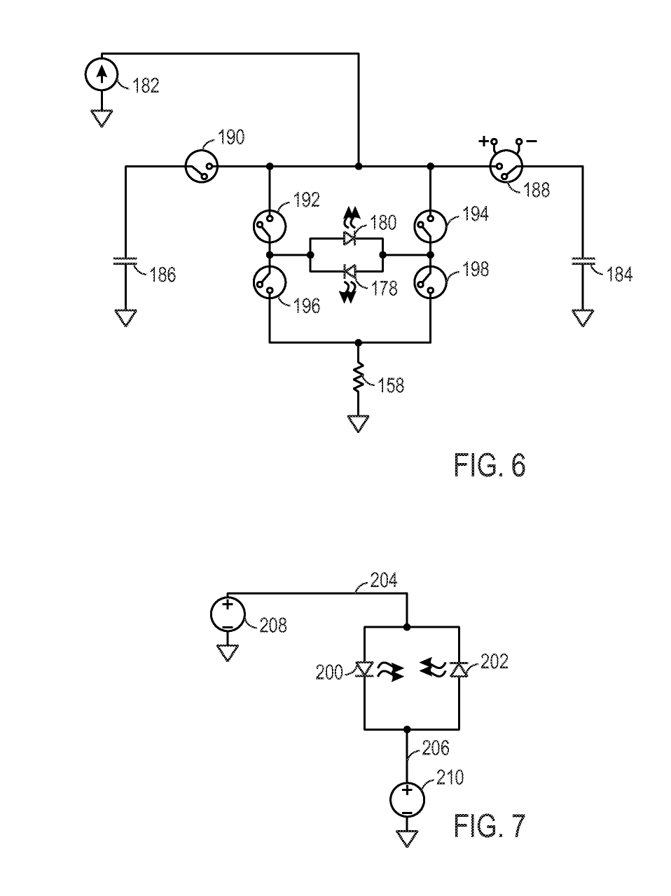

Another technique for improving a LED drive device that uses back to back LEDs, such as sensor 114, is illustrated in FIG. 6. The circuitry illustrated in FIG. 6 allows better filtering, improved rise and fall times, improved efficiency, and improved signal to noise ratios (SNRs), particularly when switching LED drives are implemented.

As previously noted, emitter 116 of the sensor 114 may include back to back LEDs, whereby the two LEDs are different (e.g., emit different wavelengths of light). One LED may be a RED LED 178 while the other may be an IR LED 180. Because the two LEDs 178 and 180 output different wavelengths of light, they are also intrinsically different in forward voltage. Thus, when a current source 182 passes a fixed current through each LED 178 and 180, the current source matches the forward voltage for the individual LED 178 or 180 and no voltage when the respective LED 178 or 180 is turned off. This may typically reduce the amount of filtering on the output of the LEDs 178 and 180 because typically only a small capacitor may swing through zero volts to the forward voltage of LED 178 to the forward voltage of LED 180 in the amount of time available.

Accordingly, to aid in the filtering of the output of the LEDs 178 and 180, two separate capacitors may be implemented, i.e., a RED capacitor 184 and an IR capacitor 186 that each correspond to the RED LED 178 and the IR LED 180, respectively. Additionally, each capacitor 184 and 186 may only be connected to its respective LED 178 or 180 during an individual on times of the respective LED 178 or 180. This may be accomplished via two switches 188 and 190 that control when the respective capacitors 184 and 186 are connected to their respective LEDs 178 and 180. Additionally, switches 192, 194, 196, and 198 may operate to control the on and off time of the LEDs 178 and 180. For example, switches 192 and 198 may be closed when the IR LED 180 is in the on state, while simultaneously switches 194 and 196 may be open when the IR LED 180 is in the on state (rendering the RED LED 178 in the off state). Similarly, switches 192 and 198 may be open when the IR LED 180 is in the off state, while simultaneously switches 194 and 196 may be closed when the IR LED 180 is in the on state (thus placing the RED LED 178 in the on state). In this manner, the switches 192, 194, 196, and 198 may control the on and off time of the LEDs 178 and 180.

Because each of the LEDs 178 and 180, as illustrated in FIG. 6, will have a fixed voltage, customized capacitors 184 and 186 can be chosen to allow for tailored filtering of each of the signals generated by the LEDs 178 and 180. Additionally, the customization of the capacitors 184 and 186 with the respective LEDs 178 and 180 allows for more rapid rise times, which allows processor 122 to utilize more samples of the wave form generated by the LEDs 178 and 180 and, thus, improve the SNR. An additional benefit includes rapid fall times when the LEDs 178 and 180 are disconnected during dark (off) time which, again, allows for a less noisy (cleaner) signal to be present (improving SNR). It should also be noted that the system illustrated in FIG. 6 may be operated in conjunction with the regulator circuitry of FIG. 3.

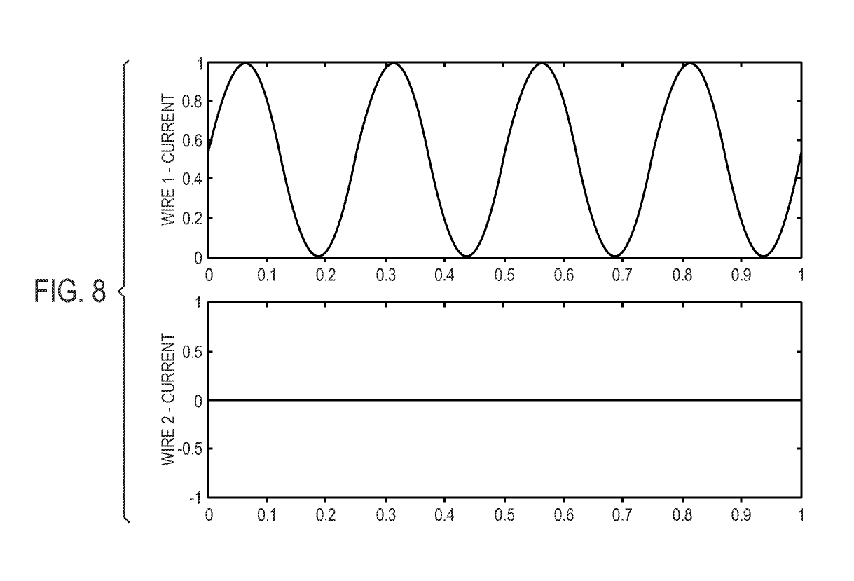

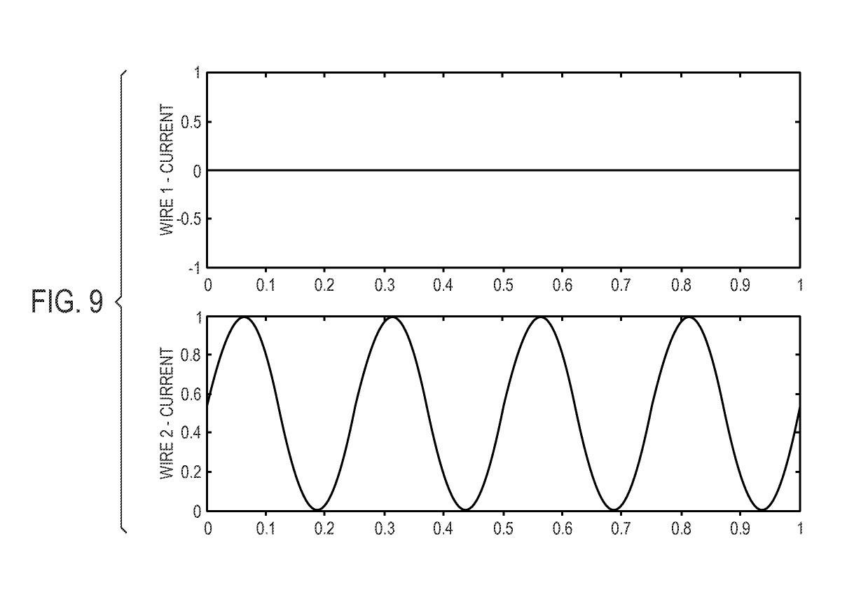

Additional techniques may be applied to present embodiments to reduce noise. For example, FIG. 7 illustrates a differential drive scheme for the sensor 114, which reduces emissions, noise, and crosstalk. As previously discussed, pulse oximeter 100 may operate by driving current through LEDs 200 (RED) and 202 (IR) and measuring light which passes through a patient 117 to be received by a photo detector 118. Typically, these LEDs 200 and 202 are driven by driving a current through one wire and ground on a second wire. Additionally, similar to the system illustrated in FIG. 6, the RED LED 200 may be in a back to back configuration with the IR LED 202 in the emitter 116 of the sensor 114, such that the current drive and ground wires are reversed to drive RED or IR light. FIG. 8 illustrates how a sinusoidal current can be driven through one LED (e.g., LED 200) from one wire (wire 1) to a second wire (wire 2). Similarly, FIG. 9 illustrates how a sinusoidal current can be driven through another LED (e.g., LED 202) from a second wire (wire 2) to a first second wire (wire 1). Analog switches are typically utilized in this scheme (e.g., switches 192, 194, 196, and 198) allowing voltage and current changes in one wire while the other wire remains grounded. However, this technique may be susceptible to noise and produce crosstalk along adjacent detector wires of the photo detector 118.

As such, returning to FIG. 7, a differential drive scheme for LEDs 200 and 202 is illustrated. Any suitable drive waveform may be utilized in conjunction with the system of FIG. 7, including (but not limited to) a square wave or sine wave. As illustrated in FIG. 7, Instead of grounding one side of the LEDs 200 and 202, the present embodiment allows for differential driving of each of two paths (wires) 204 and 206 in a complimentary manner via a first source 208 and a second source 210. In this manner, switches (e.g., switches 192, 194, 196, and 198) may be omitted as there is no need to switch either of the LEDs 200 and 202 to ground. For example, the sources 208 and 210 may be set such that both paths (wires) 204 and 206 may have a positive voltage, such that only a positive supply rail is needed (although bipolar supplies as sources 208 and 210 may be used). Likewise, the difference between the two paths (wires) 204 and 206 can be either positive or negative, allowing either of the LEDs 200 and 202 to be illuminated.

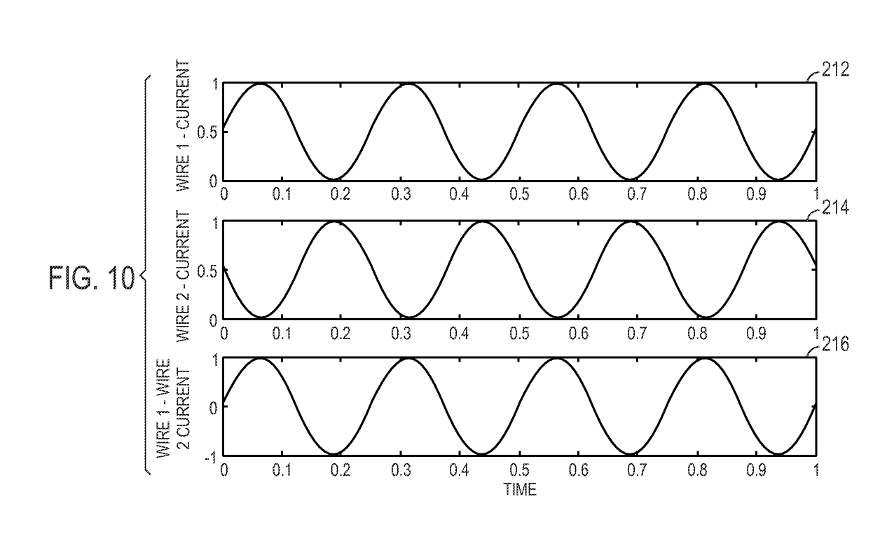

For example, as shown in FIG. 10, waveform 212 illustrates a sinusoidal current through LED 200 when path (wire) 204 has a greater voltage than path (wire) 206. Similarly, current flows through LED 202 when path (wire) 206 has greater voltage than path (wire) 204, as illustrated in waveform 214. Accordingly, the difference between the two paths (wires) 204 and 206 (illustrated in waveform 216) alternates between driving LED 200 and LED 202 during the positive and negative illustrated sections of waveform 216. These two paths (wires) 204 and 206 may be a twisted pair coupling the monitor 102 to the sensor 114 such that the voltage and current changes in both wires simultaneously, thus reducing interference and susceptibility to noise. This may allow for less shielding, for example, to be utilized in cable 115 while still allowing for a reduction in signal transmission errors (i.e., noise due to transmission of signals between the monitor 102 and the sensor 114).

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of blood oxygen saturation, but these techniques may also be utilized for the measurement and/or analysis of other blood constituents. For example, using the same, different, or additional wavelengths, the present techniques may be utilized for the measurement and/or analysis of carboxyhemoglobin, met-hemoglobin, total hemoglobin, fractional hemoglobin, intravascular dyes, and/or water content. Rather, the various embodiments may cover all modifications, equivalents, and alternatives falling within the spirit and scope of the disclosure as defined by the following appended claims.

What is claimed is:

1. An oximetry device comprising:
a controlled switch configured to transmit and resist a current flow;
an energy storage element configured to store energy in response to the current flow being transmitted and discharge the stored energy to a diode as a diode current in response to the controlled switch resisting the current flow;
a sense resistor configured to receive diode current and generate a sensed voltage based on the diode current; and
a comparator configured to activate the controlled switch to transmit the current flow and deactivate the controlled switch to resist the current flow based upon the sensed voltage.

2. The oximetry device of claim 1, wherein the comparator is configured to receive a set point and activate the controlled switch based upon a comparison between the set point and the sensed voltage.

3. The oximetry device of claim 2, comprising a digital to analog converter configured to generate and transmit the set point to the comparator.

4. The oximetry device of claim 3, comprising a processor, wherein processor is configured to calibrate the digital to analog converter to generate the set point.

5. The oximetry device of claim 1, comprising a light emitting diode configured to receive the diode current, wherein the light emitting diode is in series with the sense resistor.

6. The oximetry device of claim 5, comprising a filter coupled in parallel with the light emitting diode, wherein the filter is configured to filter the diode current.

7. The oximetry device of claim 5, comprising a processor configured to receive a first set of values related to sampled measurements of the light emitting diode, receive a second set of values related to sampled measurements of the diode current, and generate a correction value based on the first set of values and the second set of values.

8. The oximetry device of claim 1, wherein the sense resistor comprises a first resistor having a first resistance and a second resistor having a second resistance such that a resistance of the sense resistor can be altered.

9. The oximetry device of claim 1, wherein the sense resistor comprises a first resistor having a first resistance and a transistor, wherein the first resistor and the transistor are coupled in series.

10. An oximetry system comprising:
a processor configured to:
receive an indication of a diode current passing through a light emitting diode;
compare the indication of the diode against a set point;
generate an activation signal based on the comparison of the indication of the diode current with the set point; and
transmit the activation signal, wherein the activation signal is configured to activate a controlled switch to control current flow from the controlled switch.

11. The oximetry system of claim 10, comprising the controlled switch, wherein the controlled switch is configured to resist the current flow based on the activation signal.

12. The oximetry system of claim 11, comprising an energy storage element configured to store energy in response to the current flow being transmitted from the controlled switch and discharge the stored energy as the diode current in response to the controlled switch resisting the current flow.

13. The oximetry system of claim 10, comprising a sense resistor configured to generate the indication of the diode current;

14. The oximetry system of claim 13, wherein the sense resistor comprises a first resistor having a first resistance and a second resistor having a second resistance, wherein the first resistor and second resistor are coupled in parallel via a switch.

15. The oximetry system of claim 13, wherein the sense resistor comprises a first resistor having a first resistance and a transistor, wherein the first resistor and the transistor are coupled in series.

16. A oximetry device comprising:
a current source configured to provide a diode current;
a first light emitting diode (LED) configured to receive the diode current and transmit light of a first wavelength in response to receiving the diode current;
a first filter coupled in parallel with the first LED, wherein the first filter is configured to filter the diode current;
a second LED configured to receive the diode current and transmit light of a second wavelength in response to receiving the diode current;
a second filter coupled in parallel with the second LED, wherein the second filter is configured to filter the diode current;
a sense resistor configured to receive diode current subsequent to its passing through either of the first LED or the second LED and generate a sensed voltage based on the diode current, wherein the current source is configured to provide the diode current based on the sensed voltage.

17. The oximetry device of claim 16, wherein the first filter is selected based on the operational characteristics of the first LED.

18. The oximetry device of claim 16, wherein the second filter is selected based on the operational characteristics of the second LED.

19. The oximetry device of claim 16, wherein the current source is configured to supply the diode current based on the operation of a controlled switch.

20. The oximetry device of claim 19, wherein the operation of the controlled switch is based upon an activation signal generated as a pulsed wave by a pulse width modulator.
